(19) United States
(12) Patent Application Publication
(10) Pub. No.: US 2024/0335277 A1
(43) Pub. Date: Oct. 10, 2024

(54) FULL-VISUAL RANGE INTRAOCULAR LENS

(71) Applicant: WUXI VISION PRO LTD., Wuxi (CN)

(72) Inventors: Shasha LI, Wuxi (CN); Xiugao LIAO, Wuxi (CN); Zhenyu FENG, Wuxi (CN)

(73) Assignee: WUXI VISION PRO LTD., Wuxi (CN)

(21) Appl. No.: 18/746,048

(22) Filed: Jun. 18, 2024

Related U.S. Application Data
(63) Continuation of application No. PCT/CN2023/132377, filed on Nov. 17, 2023.

(30) Foreign Application Priority Data
Nov. 22, 2022 (CN) 202211470181.5

(51) Int. Cl. A61F 2/16

(52) U.S. Cl. CPC A61F 2/1618; A61F 2/164; A61F 2002/169

(57) ABSTRACT

A full-visual range intraocular lens includes an optical body, a first supporting loop and a second supporting loop, where the optical body, the first supporting loop and the second supporting loop are of integral structure and are integrally formed from the same material, and the optical body is positioned between the first supporting loop and the second supporting loop; the optical body includes two optical surfaces, which being spherical or aspherical, and one of the optical surfaces has a diffraction structure for modulating incident optical-field distribution. The full-visual range intraocular lens adjusts the incident optical-field distribution by means of a diffraction structure, reducing sharp points on the whole optical surface, effectively reducing glare, reducing chromatic aberration, and enabling to see more clearly on the visual range between focal points, so as to bring better visual experience to patients.

### CROSS REFERENCE TO THE RELATED APPLICATIONS

This application is the continuation application of International Application No. PCT/CN2023/132377, filed on Nov. 17, 2023, which is based upon and claims priority to Chinese Patent Application No. 202211470181.5, filed on Nov. 22, 2022, the entire contents of which are incorporated herein by reference.

### TECHNICAL FIELD

The present invention relates to the technical field of optical elements, and in particular to a full-visual range intraocular lens.

### BACKGROUND

Intraocular lens (IOL) refers to an optical element made of synthetic materials, which has become a common medical device to replace the natural lens with turbid lesion, so as to help patients suffering from cataract obtain postoperative vision. Multifocal intraocular lens is a kind of intraocular lens, because multifocal intraocular lens (IOL) can provide two or more focal points (e.g., trifocal IOL provides far-vision, mid-vision and near-vision), and it is widely used for implantation in cataract surgery to replace natural lens of lesion in the eyes of patients. The multifocal IOL can be divided into two categories of diffractive and refractive types, where the surface of the diffractive multifocal IOL includes a plurality of small step gratings with concentric rings to diffract light in several directions, forming a plurality of focal points; the surface of the refractive multifocal IOL may include a plurality of concentric rings of spherical or aspherical surfaces of different curvatures, or a plurality of sectors of spherical or aspherical surfaces of different curvatures, or a combination of both. However, a traditional multifocal IOL still has a problem of discontinuous visual range. There is obvious visual decline or even breakpoint among several focal points. The visual decline or breakpoint can lead to the discontinuity of visual range and the decrease of patient's ability to recognize moving objects.

In summary, how to design a full-visual range intraocular lens to provide a segment of continuous vision for a patient and improve the dynamic vision of the patient is a technical problem to be solved by a person skilled in the art.

### SUMMARY

The present invention provides a full-visual range intraocular lens for solving the problem of discontinuous visual range of diffractive multifocal intraocular lens, improving light energy utilization rate, reducing sharp points on the whole optical surface, effectively reducing glare, reducing chromatic aberration, making the full-visual range more clear among focal points, and giving the patients a better visual experience.

To achieve the foregoing purpose, the following technical solutions of the present invention are used:

A full-visual range intraocular lens including an optical body, where the optical body includes two optical surfaces, said optical surfaces being spherical or aspherical, one of the optical surfaces having a diffraction structure for modulating incident optical-field distribution;
the method for determining the two optical surfaces of said optical body includes:
establishing an arbitrary spatial rectangular coordinate system with a vertex of the optical surface as an origin O and an optical axis as an axis Z, where a coordinate axis X of the coordinate system and a coordinate axis Y of the coordinate system are tangent to said optical surface, and a surface shape of said optical surface satisfies the following equation on a Y-Z plane:

$$Z ( y ) = \frac{{cy}^{2}}{1 + \sqrt{1 - ( 1 + K ) {c}^{2} {y}^{2}}} + {\sum }_{i = m}^{n} {A}_{2 i} {y}^{2 i} \qquad ( 1 )$$

where Z (y) is a curve expression of the optical surface on a two-dimensional coordinate system plane Y-Z, c is a reciprocal of a curvature radius of a basic spherical surface of said optical surface, y is a vertical distance from any point on said curve to the coordinate axis Z, A₂ᵢ is a coefficient of higher-order terms of the optical surface, m and n are both integers not less than 1 and n>m, and K is a coefficient of the optical surface; when K and A₂ᵢ are 0, Z (y) is a spherical equation;
a method of diffraction structure modulating incident optical-field distribution is as follows:
the diffraction structure includes a diffraction ring band structure and a discrete diffraction phase point; firstly, determining a fixed focal point using the diffraction ring band structure, i.e., for a single-focus diffraction element having a diffraction ring band structure, and characterizing the phase thereof by a diffraction phase function Φ:

$$Φ ( x , y ) = {ρ}_{1} {x}^{2} + {ρ}_{2} {x}^{4} \qquad ( 2 )$$

where x and y represent longitudinal and transverse coordinates, respectively, in millimeters (mm); p₁ and p₂ are various coefficients of the diffraction phase; the phase function T (@) of the diffraction ring band structure is obtained after compressing the diffraction phase function with a period of 2π:

$$T ( Φ ) = Φ - int ( \frac{Φ}{2 π} ) * 2 π \qquad ( 3 )$$

where int ( ) represents a rounding function;
secondly, introducing a discrete diffraction phase point on a sub-wavelength order, where according to Fermat principle, a path of light propagation is the path where an optical length takes the extreme value, which is the maximum value, the minimum value or an inflection point of a function, and is characterized by the formula:

$$OPL = \int \text{?} n ( \vec{s} ) ds = \frac{λ}{2 π n} \int \text{?} d Φ ( \vec{s} ) ds \qquad ( 4 )$$

$$\text{?} \text{indicates text missing or illegible when filed}$$

where OPL is the optical length; \vec{S} is displacement of light and has a coordinate of \vec{s}(x,y), and |\vec{S}|\sqrt{(x²+y²)}, n(\vec{s}) is a refractive index of the medium at each displacement through which light passes; λ is a wavelength of light; Φ(\vec{s}) is a diffraction phase at each displacement through which light passes; when adding a plurality of discrete diffraction phase points Φ(\vec{s}) of sub-wavelength orders with different positions and sizes in the single-focus diffraction element, formula (4) is converted into the following form:

$$OPL = \frac{λ}{2 π n} \int \text{?} Φ ( \vec{s} ) ds + Φ ( \vec{{s}^{′}} ) \qquad ( 5 )$$

$$\text{?} \text{indicates text missing or illegible when filed}$$

in the above formula, s′ is a coordinate of the discrete diffraction phase point in mm; by adjusting the number, position and size of the discrete diffraction phase point Φ(\vec{s′}) to change the optical length OPL, a number of discrete diffraction phase points of different sub-wavelength orders are introduced in the same diffraction ring band structure region, so that the single-focus diffraction element extends from focusing to only one focal point to a range of focal depth with a clear full-visual range, and the method of diffraction structure modulating incident optical-field distribution is applied to the optical body, so that the optical body has the effect of full-visual range.

In the foregoing solution, by introducing the discrete diffraction phase point of sub-wavelength order, the incident optical-field distribution is modulated, the optical length is changed, and then the focal point position within a certain range is changed, and the focal depth range is extended; if a plurality of discrete diffraction phase points with different sub-wavelength orders are introduced in the same diffraction structure region, the single-focus diffraction element can be extended from focusing to only one focal point to a range of focal depth which can be imaged continuously and clearly, and two or even more focal points can be continuously combined to provide a continuous vision for the patient, so as to realize the leap from point vision to continuous vision, improve the dynamic vision of the patient and improve the light energy utilization rate.

Further, said optical body is a lenticular/meniscus lens having a clear optic diameter of 5.5 to 6.5 mm and a central thickness of 0.4 to 1.25 mm.

Further, there are two or more additional focal points of said optical body, the range of the additional dioptric power is +1.5D to +5D, and the full-visual range is +1.5D to +5D. Said optical body may be a trifocal intraocular lens.

Further, the intraocular lens further includes a first supporting loop and a second supporting loop with said optical body positioned between the first supporting loop and the second supporting loop.

Further, said optical body, the first supporting loop and the second supporting loop are of integral structure and are integrally formed from the same material.

Further, the thickness of each of said first supporting loop and said second supporting loop is 0.15 to 0.35 mm.

Further, the surfaces of said first supporting loop and said second supporting loop are both provided with oblique sawtooth grooves or protruding abrazine.

Further, a width of said oblique sawtooth groove or protruding abrazine is 0.2 to 1.0 mm.

Further, a height of said oblique sawtooth groove or protruding abrazine is greater than 40 μm.

Further, an included angle α between an oblique edge of said oblique sawtooth and a plane to which the first supporting loop and the second supporting loop belong is between −20° and +20°.

A full-visual range intraocular lens is prepared through the following steps of design:

(1) Optical design: dividing an intraocular lens into m+n regions in a concentric ring manner, where m is the number of fixed-focus regions (regions where a diffraction ring band structure is located), m+1 is the number of focal points, and n is the number of continuous regions (regions where discrete diffraction phase points are located) (for example, designing a trifocal intraocular lens, and continuously connecting two of the focal points, so that the intraocular lens will be divided into 3-1+1=3 regions); where the continuous region has a full-visual range effect, and the area of the fixed-focus region accounts for 50%-70% of the whole area of the diffraction structure, and the area of the continuous region accounts for 30%-50% of the whole area of the diffraction structure.

Design of fixed-focus region: according to the design of traditional bifocal intraocular lens, firstly determining the number of additional focal points and corresponding dioptric power, constructing an initial model in Zemax, and then optimizing to obtain the optimal expected effect, obtaining the diffraction basic parameters, and determining a polynomial expression of the diffraction phase function:

$${Φ}_{1} ( x , y ) = {ρ}_{11} {x}^{2} + {ρ}_{21} {x}^{4}$$

$${Φ}_{2} ( x , y ) = {ρ}_{12} {x}^{2} + {ρ}_{22} {x}^{4}$$

$$…$$

$${Φ}_{m} ( x , y ) = {ρ}_{1 m} {x}^{2} + {ρ}_{2 m} {x}^{4}$$

where m is a positive integer greater than or equal to 2. At the same time, the surface profiles Z_{A} and Z_{B} of the two optical surfaces of the intraocular lens are determined; after the diffraction phase function is compressed with a period of 2π, the height h_{diffraction} of the diffraction structure corresponding to each radial position x is calculated; the diffraction ring band structure is superimposed on an optical surface of the optic to obtain an actual optical surface shape Zₒᵥₑᵣₐₗₗ=Z_{basic}+h_{diffraction} of the fixed-focus region, and Z_{basic} is a refractive basal surface.

Design of continuous region: adding a plurality of discrete diffraction phase points Φ(\vec{sₙᵢ′}) of sub-wavelength orders with different positions and sizes in each continuous region according to the required dioptric power of the continuous focal point, where n and i are both positive integers greater than or equal to 1, and i is the number of discrete diffraction phase points introduced into one region; Φ(\vec{sₙᵢ′})=(xₙᵢ, yₙᵢ) is the coordinate of the discrete diffraction phase point in mm.

By adjusting the number, position and size of the discrete diffraction phase points Φ(\vec{sₙᵢ′}), introducing these discrete diffraction phase points into formula (5) to change the optical length OPL, extending the single-focus diffraction element from focusing to only one focal point to have a range of focal depths that can be imaged continuously and clearly. Similarly, after compressing these discrete diffraction phase points with a period of 2π, the height h_{continuous} of the diffraction structure corresponding to each radial position x can be calculated, and then the actual optical surface shape as Z′ₒᵥₑᵣₐₗₗ=Z_{basic}+h_{continuous} of the fixed-focus region can be obtained.

(2) Lathing a base refractive lens: according to the machining parameters of front and rear optical surfaces of optical design, a lathe program is written; an optical body is lathed with a diamond single-point cutting technology; a milling program is written to mill out the profile of optical region and loop feet with abrazine/sawtooth shape; and

(3) Polishing to obtain the intraocular lens with qualified optical surface.

Said full-visual range intraocular lens can be manufactured by firstly machining an intraocular lens disc containing a diffraction structure with a lathe, and then making the lens optical body through mechanical engraving; and the first supporting loop and the second supporting loop are made through mechanical cutting.

Compared with the prior art, the advantageous effects of the technical solution of the present invention are:
the present invention extends the focal point by introducing discrete diffraction phase points into the diffraction structure to achieve a continuous visual range, which can not only enable the patient to see clearly on the fixed focal point, but also on the visual range between focal points, so as to achieve a leap from point vision to continuous vision, and solve the problem of discontinuous visual range of the diffractive multifocal intraocular lens.

The optic of the optical body of the present invention has the effect of full-visual range (i.e., continuous visual range), which simulates a continuous zooming function of the human eyes, can clearly image a continuously moving object, improve patients' dynamic vision, and can clearly image the moving object.

In the present invention, discrete diffraction phase points are introduced to adjust the incident optical-field distribution by means of a diffraction ring band structure, reducing sharp points on the whole optical surface, effectively reducing glare and reducing chromatic aberration, so as to bring better visual experience to patients.

In the present invention, the focal points are not completely separated, and the continuous focal depth range increases the light energy utilization rate to greater than 90%, allowing patients to see more clearly in a dark environment.

A one-piece integrally formed full-visual range intraocular lens is simpler in structure than a complicated mechanically adjustable intraocular lens, so that the intraocular lens of the present invention is applicable to a complicated ocular fluid environment, has good stability and is not easy to induce secondary cataract; finally, the design of the abrazine/sawtooth surface of the supporting loops increases the resistance to supporting loop movement and avoids lens rotation within the capsular bag, and further increases postoperative stability.

### BRIEF DESCRIPTION OF THE DRAWINGS

Drawings are only for purposes of illustration and cannot to be construed as limiting the present invention. In order to better explain the present embodiment, some components in the drawings are omitted, enlarged or reduced, and do not represent the dimensions of the actual product; it will be appreciated by a person skilled in the art that certain well-known structures and descriptions thereof may be omitted from the figures.

FIG. 1 is a schematic structural diagram showing the front and side structures of the full-visual range intraocular lens of Embodiment 1 according to an embodiment of the present invention;

FIG. 2 is a schematic structural diagram showing the front and side structures of the full-visual range intraocular lens of Embodiment 3 according to an embodiment of the present invention;

FIGS. 3A-3F are graphs of a USAF1951 resolution plate test at different dioptric powers over a continuous range introduced into an eye model for Embodiment 1 according to an embodiment of the present invention;

FIGS. 4A-4K are graphs of a USAF1951 resolution plate test at different dioptric powers over a continuous range introduced into an eye model for Embodiment 2 according to an embodiment of the present invention; and

FIGS. 5A-5I are graphs of a USAF1951 resolution plate test at different dioptric powers over a continuous range introduced into an eye model for Embodiment 3 according to an embodiment of the present invention.

### DETAILED DESCRIPTION OF THE EMBODIMENTS

In order that the objectives, aspects and advantages of the embodiments of the present invention will become more apparent, a more complete description of the embodiments of the present invention will be rendered by reference to the accompanying drawings which illustrate, by way of example, some, but not all embodiments of the present invention. Based on the embodiments of the present application, all other embodiments obtained by a person skilled in the art without involving any inventive effort fall within the scope of protection of the Invention.

### Embodiment 1

The present embodiment provides a full-visual range intraocular lens including an optical body 1, where the optical body 1 includes two optical surfaces, said optical surfaces being spherical or aspherical, one of the optical surfaces having a diffraction structure for modulating incident optical-field distribution;
the method for determining the two optical surfaces of the optical body 1 includes:
establishing an arbitrary spatial rectangular coordinate system with a vertex of the optical surface as an origin O and an optical axis as an axis Z, where a coordinate axis X of the coordinate system and a coordinate axis Y of the coordinate system are tangent to the optical surface, and a surface shape of said optical surface satisfies the following equation on a Y-Z plane:

$$Z ( y ) = \frac{{cy}^{2}}{1 + \sqrt{1 - ( 1 + K ) {c}^{2} {y}^{2}}} + {\sum }_{i = m}^{n} {A}_{21} {y}^{2 i} \qquad ( 1 )$$

where Z (y) is a curve expression of the optical surface on a two-dimensional coordinate system plane Y-Z, c is a reciprocal of a curvature radius of a basic spherical surface of said optical surface, y is a vertical distance from any point on said curve to the coordinate axis Z, A₂ᵢ is a coefficient of higher-order terms of the optical surface, m and n are both integers not less than 1 and n>m, and K is a coefficient of the optical surface; when K and A₂ᵢ are 0, Z (y) is a spherical equation;
a method of diffraction structure modulating incident optical-field distribution is as follows:
the diffraction structure includes a diffraction ring band structure and a discrete diffraction phase point; firstly, determining a fixed focal point using the diffraction ring band structure, i.e., for a single-focus diffraction element having a diffraction ring band structure, and characterizing the phase thereof by a diffraction phase function Φ:

$$Φ ( x , y ) = {ρ}_{1} {x}^{2} + {ρ}_{2} {x}^{4} \qquad ( 2 )$$

where x and y represent longitudinal and transverse coordinates, respectively, in millimeters (mm); p₁ and p₂ are various coefficients of the diffraction phase; the phase function T (Φ) of the diffraction ring band structure is obtained after compressing the diffraction phase function with a period of 2π:

$$T ( Φ ) = Φ - int ( \frac{Φ}{2 π} ) * 2 π \qquad ( 3 )$$

where int ( ) represents a rounding function;
secondly, introducing a discrete diffraction phase point on a sub-wavelength order, where according to Fermat principle, a path of light propagation is the path where an optical length takes the extreme value, which is the maximum value, the minimum value or an inflection point of a function, and is characterized by the formula:

$$OPL = \int \text{?} n ( \vec{s} ) ds = \frac{λ}{2 π n} \int \text{?} Φ ( \vec{s} ) ds \qquad ( 4 )$$

$$\text{?} \text{indicates text missing or illegible when filed}$$

where OPL is the optical length; \vec{S} is displacement of light and has a coordinate of \vec{s}(x,y), and |\vec{s}|=\sqrt{(x²+y²)}; n(\vec{s}) is a refractive index of the medium at each displacement through which light passes; λ is a wavelength of light; Φ(\vec{s}) is a diffraction phase at each displacement through which light passes; when adding a plurality of discrete diffraction phase points Φ(\vec{s′}) of sub-wavelength orders with different positions and sizes in the single-focus diffraction element, formula (4) is converted into the following form:

$$OPL = \frac{λ}{2 π n} \int \text{?} Φ ( \vec{s} ) ds + Φ ( \vec{{s}^{′}} ) \qquad ( 5 )$$

$$\text{?} \text{indicates text missing or illegible when filed}$$

in the above formula, s′ is a coordinate of the discrete diffraction phase point in mm; by adjusting the number, position and size of the discrete diffraction phase point Φ(\vec{s′}) to change the optical length OPL, a number of discrete diffraction phase points of different sub-wavelength orders are introduced in the same diffraction ring band structure region, so that the single-focus diffraction element extends from focusing to only one focal point to a range of focal depth with a clear full-visual range, and the method of diffraction structure modulating incident optical-field distribution method is applied to the optical body 1, so that the optical body 1 has the effect of full-visual range.

### Embodiment 2

Specifically, on the basis of Embodiment 1, the solution will be described in conjunction with a specific embodiment to further embody the technical effects of the solution. Specifically:

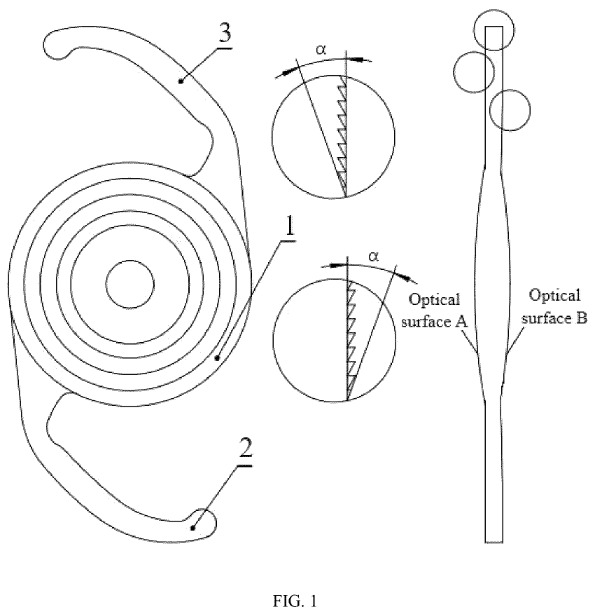

In order to facilitate understanding, with reference to FIG. 1, the present invention provides an embodiment of a full-visual range intraocular lens including an optical body 1, a first supporting loop 2 and a second supporting loop 3, where the optical body 1, the first supporting loop 2 and the second supporting loop 3 are of an integral structure and are integrally formed from the same material, and said optical body 1 is composed of two optical surfaces (an optical surface A and an optical surface B), and said optical surfaces are spherical or aspherical, where the optical surface A has a diffraction structure for modulating the incident optical-field distribution.

The diffraction structure is embossed on an optical surface of the optical body 1 by means of lathing, where the diffraction structure is selected to be superimposed on the optical surface A; the surfaces of the first supporting loop 2 and the second supporting loop 3 are both provided with oblique sawtooth grooves; the height of the oblique sawtooth is greater than 40 μm, the width of the oblique sawtooth groove is 0.2 mm, and the included angle α between the oblique edge of the oblique sawtooth and the plane to which the supporting loop belongs is 20°. The supporting loops include the first supporting loop 2 and the second supporting loop 3.

A lenticular lens has a clear optic of the optical body 1 of 6.0 mm in diameter and a central thickness of 0.67 mm; the thicknesses of said first supporting loop 2 and said second supporting loop 3 are both 0.15 mm; the optical body 1 is made of a hydrophobic polyacrylate having a refractive index of 1.544 and a dispersion coefficient of 45 to 55.

The preparation method for the full-visual range intraocular lens of the present embodiment includes:
(1) Design solution: a trifocal intraocular lens is designed, 20D for far focal point, 2D for middle focal point and 3D for near focal point, and the middle focal point and the near focal point are continued; and
(2) Optical design: the fixed-focus region and the continuous region are divided through each focal length of proposed multifocal points, which are 50 mm (20D), 45.45 mm (22D) and 43.48 mm (23D), as two fixed-focus regions and one continuous region. The total area of the fixed-focus region is 70%, and the total area of the continuous region is 30%. The initial model is built in Zemax, and optimized to obtain the parameters of the optical surface A and the optical surface B of the optical body 1, in which the base radius is 20.5 mm and K=−11.68. The phase functions of the two fixed-focus regions are determined according to the phase coefficients:

$${Φ}_{1} ( x , y ) = - 101.13 {x}^{2} - 0.786 x \text{?}$$

$${Φ}_{2} ( x , y ) = - 151.7 {x}^{2} - 1.48 {x}^{4}$$

$$\text{?} \text{indicates text missing or illegible when filed}$$

Further, according to the required two continuous focal points 22D and 23D, it is decided to add five discrete diffraction phase points, which are uniformly distributed in a continuous region and have a size of intermediate values of Φ₁ and Φ₂.

(3) Lathing the base refractive lens: according to the designed optic parameters, a lathe program is written; a disc intraocular lens is lathed by diamond single-point cutting technology; a milling program is written to mill out the profile of the optic and the supporting loop feet with an abrazine shape.

(4) Polishing to obtain the intraocular lens with qualified optical surface.

(5) Analyzing tests in an eye model.

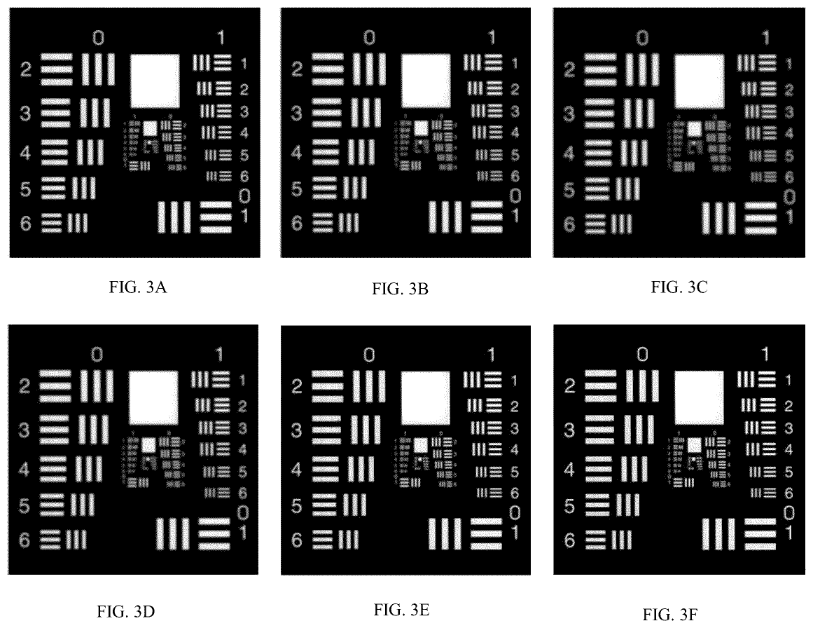

A full-visual range intraocular lens IOL of Embodiment 1 is introduced into the eye model required in ISO11979-2, and a graph of USAF1951 resolution plate test with an interval of 0.2D from 22D to 23D is obtained by means of the optical device test; as shown in FIGS. 3A-3F, it can be seen that the full-visual range intraocular lens IOL can achieve clear visual effect between 22D and 23D; where FIG. 3A: 22D; FIG. 3B: 22.2D; FIG. 3C: 22.4D; FIG. 3D: 22.6D; FIG. 3E: 22.8D; FIG. 3F: 23D.

In view of the problem that a full-visual range intraocular lens IOL in the prior art has insufficient visual range, obvious visual decline or even breakpoint among several focal points, and the visual decline or breakpoint leading to discontinuous visual range, resulting in insufficient ability of the patient to recognize moving objects, in the present invention, by introducing a discrete diffraction phase point, modulating the incident optical-field distribution, allowing two or even more focal points to be continuous, and measuring MTF values in a continuous range which are all greater than 0.13 in a simulated eye model, it is able to provide a patient with continuous clear vision of 0.6 or more, and achieve a leap from point vision to continuous vision, improving the patient's dynamic vision, clearly imaging the moving objects, and improving the patient's quality of life, at the same time, it has the effects of reducing glare, reducing chromatic aberration and improving light energy utilization rate.

It should be noted that the optic of the optical body 1 of the present invention is a bifocal, trifocal, or multi-intersection optic. The present invention has a better improvement effect on a trifocal IOL for a problem that an obvious discontinuity of visual range in the range from mid-vision to near-vision.

### Embodiment 3

Specifically, on the basis of Embodiment 1, the solution will be described in conjunction with a specific embodiment example to further embody the technical effects of the solution. Specifically:

A full-visual range intraocular lens including an optical body 1, a first supporting loop 2 and a second supporting loop 3, where the optical body 1 includes an optical surface A, an optical surface B and a diffraction structure on the optical surface A. The optical main body 1, the first supporting loop 2 and the second supporting loop 3 are of an integral structure and are formed integrally from the same material; the diffraction structure is embossed on an optical surface of the optical body 1 by means of lathing, where the diffraction structure is selected to be superimposed on the optical surface A.

The surfaces of the first supporting loop 2 and the second supporting loop 3 are provided with several oblique sawtooth grooves, and the width of the oblique sawtooth grooves is 0.2 mm, and an included angle α between the oblique edges of the oblique sawtooth and the plane to which the supporting loops belong is 20°; the height of the oblique sawtooth is greater than 40 μm.

A lenticular lens has a clear optic of the optical body 1 of 5.5 mm in diameter and a central thickness of 0.6 mm; the thicknesses of the first supporting loop 2 and the second supporting loop 3 are both 0.15 mm; the optical body 1 is made of a hydrophobic polyacrylate having a refractive index of 1.544 and a dispersion coefficient of 45 to 55.

The preparation method for the full-visual range intraocular lens of the present embodiment includes:
(1) Design solution: a trifocal intraocular lens is designed, 15D for far focal point, 2D for middle focal point and 4D for near focal point, and the middle focal point and the near focal point are continued; and
(2) Optical design: the fixed-focus region and the continuous region are divided through each focal length of proposed multifocal points, which are 66.67 mm (15D), 58.82 mm (17D), and 52.63 mm (19D), as two fixed-focus regions and one continuous region. The total area of fixed-focus region is 50%, and the total area of continuous region is 50%. The initial model is built in Zemax, and optimized to obtain the parameters of the optical surface A and the optical surface B of the optical body 1, in which the base radius is 31.57 mm and K=−19.74. The phase functions of the two fixed-focus regions are determined according to the phase coefficients:

$${Φ}_{1} ( x , y ) = - 101.2 {x}^{2} + 0.72 x \text{?}$$

$${Φ}_{2} ( x , y ) = - 202.22 {x}^{2} + 1.424 x \text{?}$$

$$\text{?} \text{indicates text missing or illegible when filed}$$

Further, according to the required two continuous focal points 17D and 19D, it is decided to add ten discrete diffraction phase points, which are uniformly distributed in a continuous region and have a size of intermediate values of Φ₁ and Φ₂.

(3) Lathing the base refractive lens: according to the designed optic parameters, a lathe program is written; a disc intraocular lens is lathed by diamond single-point cutting technology; a milling program is written to mill out the profile of the optic and the supporting loop feet with an abrazine shape.

(4) Polishing to obtain the intraocular lens with qualified optical surface.

(5) Analyzing tests in an eye model

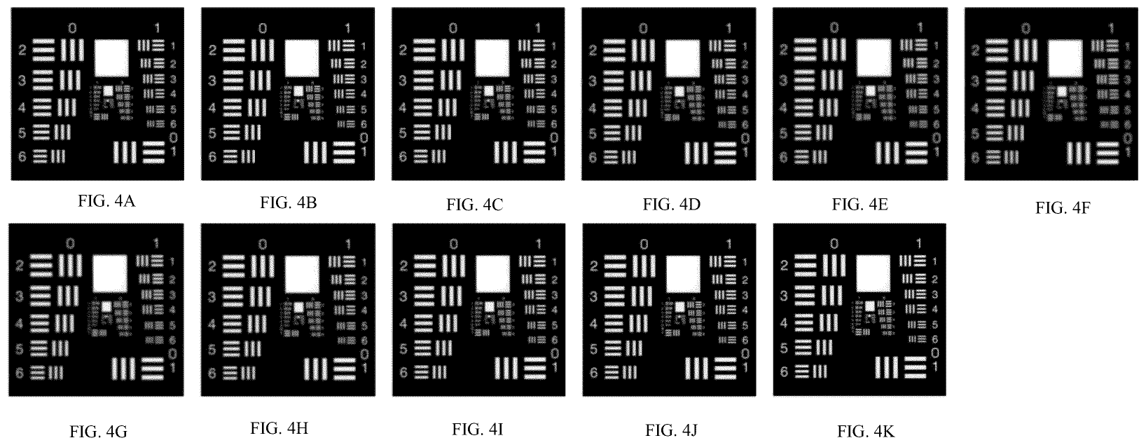

The intraocular lens IOL of the present Embodiment is introduced into the eye model required in ISO11979-2, and a graph of USAF1951 resolution plate test with an interval of 0.2D between the middle focal point (17D) and the near focal point (19D) is obtained by means of an optical device test; as shown in FIGS. 4A-4K, it can be seen that the full-visual range intraocular lens IOL can achieve clear visual effect between 17D and 19D, where FIG. 4A: 17D; FIG. 4B: 17.2D; FIG. 4C: 17.4D; FIG. 4D: 17.6D; FIG. 4E: 17.8D; FIG. 4F: 18D; FIG. 4G: 18.2D; FIG. 4H: 18.4D; FIG. 4I: 18.6D; FIG. 4J: 18.8D; FIG. 4K: 19D.

### Embodiment 4

Specifically, on the basis of Embodiment 1, the solution will be described in conjunction with a specific embodiment example to further embody the technical effects of the solution. Specifically:

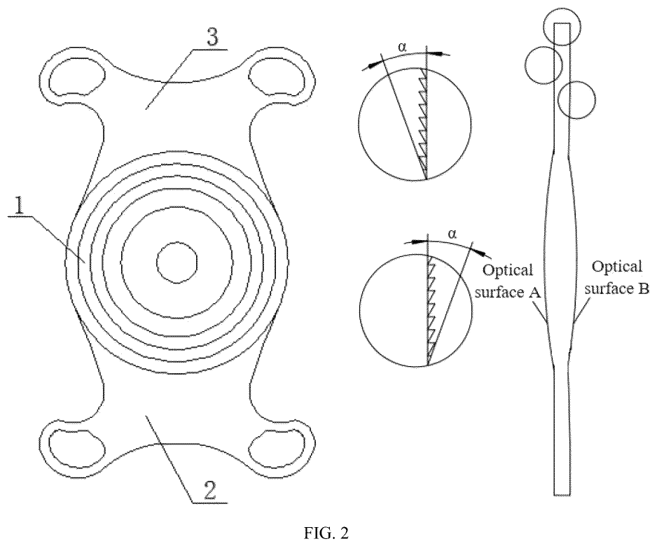

As shown in FIG. 2, a full-visual range intraocular lens including an optical body 1, a first supporting loop 2 and a second supporting loop 3, where the optical body 1 includes an optical surface A, an optical surface B and a diffraction structure on the optical surface A.

The optical main body 1, the first supporting loop 2 and the second supporting loop 3 are of an integral structure and are formed integrally from the same material; the diffraction structure is embossed on an optical surface of the optical body 1 by means of lathing, where the diffraction structure is chosen to be superimposed on the optical surface A.

The surfaces of the first supporting loop 2 and the second supporting loop 3 are provided with a plurality of oblique sawtooth grooves, where the height of the oblique sawtooth is greater than 40 μm, the width of the oblique sawtooth groove is 0.2 mm, and an included angle α between the oblique edge of the oblique sawtooth and the plane to which the supporting loop belongs is 20°.

A lenticular lens has a clear optic of the optical body 1 of 5.5 mm in diameter and a central thickness of 0.78 mm; the thicknesses of the first supporting loop 2 and the second supporting loop 3 are both 0.15 mm; the optical body 1 is made of a hydrophobic polyacrylate having a refractive index of 1.544 and a dispersion coefficient of 45 to 55.

The preparation method for the full-visual range intraocular lens of the present embodiment includes:

(1) Design solution: a trifocal intraocular lens is designed, 28D for far focal point, 2.5D for middle focal point and 4D for near focal point, and the middle focal point and the near focal point are continued; and

(2) Optical design: the fixed-focus region and the continuous region are divided through each focal length of proposed multifocal points, which are 35.71 mm (28D), 32.79 mm (30.5D), and 31.25 mm (32D), as two fixed-focus regions and one continuous region. The total area of fixed-focus region is 60%, and the total area of continuous region is 40%. The initial model is built in Zemax, and optimized to obtain the parameters of the optical surface A and the optical surface B of the optical body 1, in which the base radius is 15.75 mm and K=−8.14. The phase functions of the two fixed-focus regions are determined according to the phase coefficients:

$${Φ}_{1} ( x , y ) = - 128.746 {x}^{2} + 0.096 x \text{?}$$

$${Φ}_{2} ( x , y ) = - 206.925 {x}^{2} + 0.709 {x}^{4}$$

$$\text{?} \text{indicates text missing or illegible when filed}$$

Further, according to the required two continuous focal points 30.5D and 33D, it is decided to add eight discrete diffraction phase points, which are uniformly distributed in a continuous region and have a size of intermediate values of Φ₁ and Φ₂.

(3) Lathing the base refractive lens: according to the designed optic parameters, a lathe program is written; a disc intraocular lens is lathed by diamond single-point cutting technology; a milling program is written to mill out the profile of the optic and the supporting loop feet with an abrazine shape.

(4) Polishing to obtain the intraocular lens with qualified optical surface.

(5) Analyzing tests in an eye model

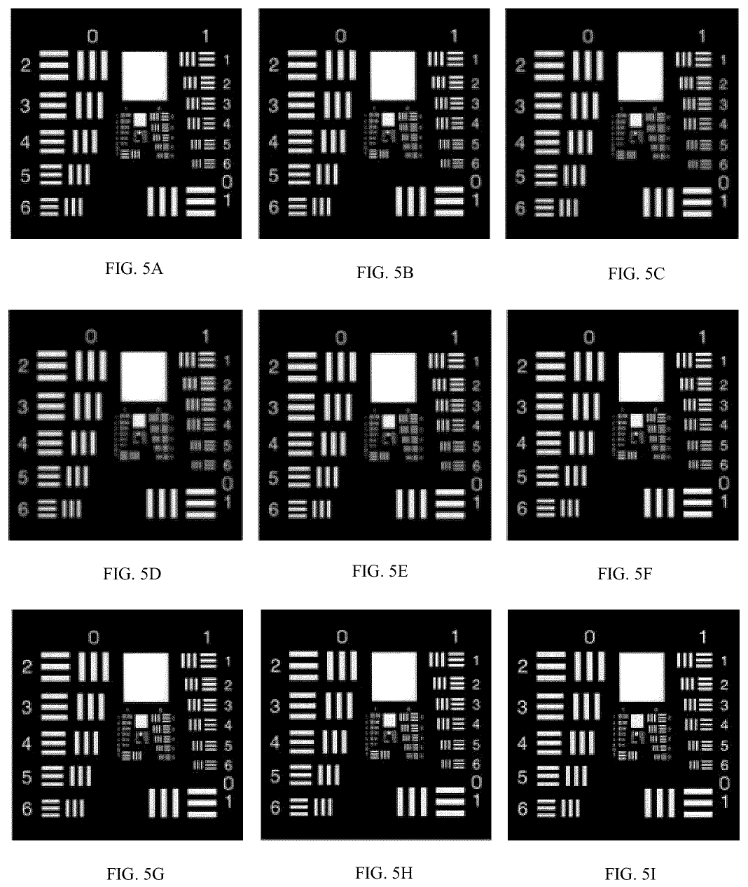

The intraocular lens IOL of the present Embodiment is introduced into the eye model required in ISO11979-2, and a graph of USAF1951 resolution plate test with an interval of 0.2D between the middle focal point (30.5D) and the near focus (32D) is obtained by means of an optical device test; as shown in FIGS. 5A-5I, it can be seen that clear visual effect between 30.5D and 32D can be achieved, where FIG. 5A: 30.5D; FIG. 5B: 30.6D; FIG. 5C: 30.8D; FIG. 5D: 31D; FIG. 5E: 31.2D; FIG. 5F: 31.4D; FIG. 5G: 31.6D; FIG. 5H: 31.8D; FIG. 5I: 32D.

It can be understood that the above-mentioned embodiments illustrate the implementation effect of the present invention by taking a trifocal intraocular lens as an example, and the method for the present invention for solving the problem of discontinuous visual range of a diffractive multifocal intraocular lens is equally applicable to other diffractive multifocal intraocular lenses.

It should be understood that the above-described embodiments of the present invention are merely illustrative of the present invention for purposes of clarity and are not intended to limit the embodiments of the present invention. It will be apparent to a person skilled in the art that various other modifications and variations can be made in the present invention without departing from the scope or spirit of the present invention. All embodiments no need to be, and cannot be, exhaustive. Thus, it is intended that the present invention cover the modifications and variations of this invention provided they come within the scope of the appended claims and their equivalents.

1. A full-visual range intraocular lens, comprising an optical body, wherein the optical body comprises two optical surfaces, the two optical surfaces being spherical or aspherical, one of the two optical surfaces having a diffraction structure for modulating an incident optical-field distribution;
a method for determining the two optical surfaces of the optical body comprises:
establishing a spatial rectangular coordinate system with a vertex of the optical surface as an origin O and an optical axis as an axis Z, wherein a coordinate axis X of the spatial rectangular coordinate system and a coordinate axis Y of the spatial rectangular coordinate system are tangent to the optical surface, and a surface shape of the optical surface satisfies the following equation on a two-dimensional coordinate system plane Y-Z:
$$Z ( y ) = \frac{{cy}^{2}}{1 + \sqrt{1 - ( 1 + K ) {c}^{2} {y}^{2}}} + {\sum }_{i = m}^{n} {A}_{21} {y}^{2 i} \qquad ( 1 )$$
wherein Z (y) is an expression of a curve of the optical surface on the two-dimensional coordinate system plane Y-Z, c is a reciprocal of a curvature radius of a basic spherical surface of the optical surface, y is a vertical distance from any point on the curve to a coordinate axis Z, A₂ᵢ is a coefficient of higher-order terms of the optical surface, m and n are both integers greater than or equal to 1 and n>m, and K is a coefficient of the optical surface; when K and A₂ᵢ are 0, Z (y) is a spherical equation;
the diffraction structure comprises a diffraction ring band structure and a discrete diffraction phase point, and a method for modulating the incident optical-field distribution by the diffraction structure comprises:
firstly, determining a fixed focal point using the diffraction ring band structure, i.e., for a single-focus diffraction element having a diffraction ring band structure, characterizing a phase of the single-focus diffraction element by a diffraction phase function @:
$$Φ ( x , y ) = {ρ}_{1} {x}^{2} + {ρ}_{2} {x}^{4} \qquad ( 2 )$$
wherein x and y represent longitudinal and transverse coordinates, respectively, in millimeters (mm); p₁ and p₂ are various coefficients of the diffraction phase function; a phase function T (Φ) of the diffraction ring band structure is obtained after compressing the diffraction phase function with a period of 2π:
$$T ( Φ ) = Φ - int ( \frac{Φ}{2 π} ) * 2 π \qquad ( 3 )$$
wherein int ( ) represents a rounding function;
secondly, introducing a discrete diffraction phase point on a sub-wavelength order, wherein according to Fermat principle, a path of light propagation is the path where an optical length takes an extreme value, wherein the extreme value is a maximum value, a minimum value or an inflection point of a function, and the path of light propagation is characterized by a formula (4):
$$OPL = \int \text{?} n ( \vec{s} ) ds = \frac{λ}{2 π n} \int \text{?} Φ ( \vec{s} ) ds \qquad ( 4 )$$
$$\text{?} \text{indicates text missing or illegible when filed}$$
wherein OPL is the optical length; \vec{S} is displacement of light and has a coordinate of \vec{s}(x,y), and |\vec{s}|=\sqrt{(x²+y²)}; n(\vec{s}) is a refractive index of a medium at each displacement through which the light passes; λ is a wavelength of light; Φ(\vec{s}) is a diffraction phase at each displacement through which the light passes; when a plurality of discrete diffraction phase points Φ(\vec{s′}) of sub-wavelength orders with different positions and sizes are added in the single-focus diffraction element, the formula (4) is converted into a formula (5):
$$OPL = \frac{λ}{2 π n} \int \text{?} Φ ( \vec{s} ) ds + Φ ( \vec{{s}^{′}} ) \qquad ( 5 )$$
$$\text{?} \text{indicates text missing or illegible when filed}$$
in the formula (5), s′ is a coordinate of the discrete diffraction phase point in mm; by adjusting a number, a position and a size of the discrete diffraction phase point Φ(\vec{s′}) to change the optical length OPL, a plurality of discrete diffraction phase points of different sub-wavelength orders are introduced in a same diffraction ring band structure region, so that the single-focus diffraction element extends from focusing to only one focal point to a range of focal depth with a clear full-visual range, and the method for modulating the incident optical-field distribution by the diffraction structure is applied to the optical body, so that the optical body has an effect of full-visual range.

2. The full-visual range intraocular lens according to claim 1, wherein the optical body is a lenticular/meniscus lens having an effective optical zone diameter of 5.5 mm to 6.5 mm and a central thickness of 0.4 mm to 1.25 mm.

3. The full-visual range intraocular lens according to claim 1, wherein there are two or more additional focal points of the optical body.

4. The full-visual range intraocular lens according to claim 1, wherein further comprising a first supporting loop and a second supporting loop, wherein the optical body is positioned between the first supporting loop and the second supporting loop.

5. The full-visual range intraocular lens according to claim 4, wherein the optical body, the first supporting loop and the second supporting loop are of an integral structure, formed integrally from a same material.

6. The full-visual range intraocular lens according to claim 4, wherein each of the first supporting loop and the second supporting loop has a thickness of 0.15 mm-0.35 mm.

7. The full-visual range intraocular lens according to claim 4, wherein each of a surface of the first supporting loop and a surface of the second supporting loop is provided with an oblique sawtooth groove or protruding abrazine.

8. The full-visual range intraocular lens according to claim 7, wherein a width of the oblique sawtooth groove or protruding abrazine is 0.2 mm-1.0 mm.

9. The full-visual range intraocular lens according to claim 8, wherein a height of the oblique sawtooth groove or protruding abrazine is greater than 40 μm.

10. The full-visual range intraocular lens according to claim 9, wherein an included angle α between an oblique edge of the oblique sawtooth groove and a plane to which the first supporting loop and the second supporting loop belong is between −20° and +20°.

11. The full-visual range intraocular lens according to claim 5, wherein each of a surface of the first supporting loop and a surface of the second supporting loop is provided with an oblique sawtooth groove or protruding abrazine.

12. The full-visual range intraocular lens according to claim 6, wherein each of a surface of the first supporting loop and a surface of the second supporting loop is provided with an oblique sawtooth groove or protruding abrazine.

13. The full-visual range intraocular lens according to claim 11, wherein a width of the oblique sawtooth groove or protruding abrazine is 0.2 mm-1.0 mm.

14. The full-visual range intraocular lens according to claim 12, wherein a width of the oblique sawtooth groove or protruding abrazine is 0.2 mm-1.0 mm.

15. The full-visual range intraocular lens according to claim 13, wherein a height of the oblique sawtooth groove or protruding abrazine is greater than 40 μm.

16. The full-visual range intraocular lens according to claim 14, wherein a height of the oblique sawtooth groove or protruding abrazine is greater than 40 μm.

17. The full-visual range intraocular lens according to claim 15, wherein an included angle α between an oblique edge of the oblique sawtooth groove and a plane to which the first supporting loop and the second supporting loop belong is between −20° and +20°.

18. The full-visual range intraocular lens according to claim 16, wherein an included angle α between an oblique edge of the oblique sawtooth groove and a plane to which the first supporting loop and the second supporting loop belong is between −20° and +20°.
